(19) United States
(12) Patent Application Publication
(10) Pub. No.: US 2023/0243441 A1
(43) Pub. Date: Aug. 3, 2023

(54) Electromagnetic Positioning Device with Position Detection

(71) Applicant: ETO MAGNETIC GmbH, Stockach (DE)

(72) Inventors: Peter Gauggel, Bodman-Ludwigshafen (DE); Bernd Hardy, Radolfzell (DE); Lutz Mantsch, Göppingen (DE); Achim Riedle, Steisslingen (DE)

(73) Assignee: ETO MAGNETIC GmbH, Stockach (DE)

(21) Appl. No.: 18/012,870

(22) PCT Filed: Jun. 22, 2021
(86) PCT No.: PCT/EP2021/066942

§ 371 Date: Dec. 23, 2022

(30) Foreign Application Priority Data
Jun. 23, 2020 (DE) 10 2020 116 482.3

(51) Int. Cl. F16K 37/00; G01S 13/02; G01S 13/32; G01N 33/28; G01N 22/00; F16K 31/06; H01F 7/18

(52) U.S. Cl. CPC F16K 37/0041; G01S 13/0209; G01S 13/32; G01N 33/28; G01N 22/00; F16K 31/0675; H01F 7/1805; H01F 7/1844

(57) ABSTRACT

The invention relates to an electromagnetic positioning device (10) having energizable stationary coil means (1), a magnetic guide element (2) associated thereto and having a core section (2a) and an anchor unit (3) moveable in relation to the guide element (2) and along an axial movement direction (L) as a reaction to an energization of the coil means (1), wherein the guide element (2) is a hollow cylinder that partially surrounds the anchor unit (3). The positioning device (10) has an ultra-wide-band sensor unit (4) which is preferably integrated in the guide element (3) and is designed for measuring the position and/or the displacement of the anchor unit (3) in the guide element (2).

The invention relates to an electromagnetic positioning device. In particular, the invention relates to an electromagnetic positioning device having optimized position detection of the anchor element.

Electromagnetic positioning devices for different tasks, such as actuators for positioning tasks, for hydraulic or pneumatic valves, in particular way solenoid valves, are sufficiently known from the state of the art. They generally have energizable stationary coil means and anchor means, which are mounted so as to be axially moveable, can be driven along an anchor axis as a reaction to an energization of the coil means and can thus be moved in relation to the casing of the positioning device, the anchor means being connected to a pestle as a positioning element or to a valve coil component group, for example for switching pneumatic or hydraulic way solenoid valves.

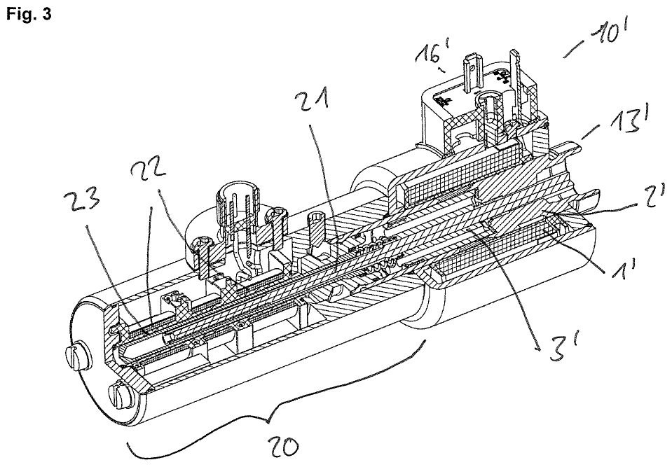

Also known is an associated position detection and/or displacement measurement unit of such devices in particular for hydraulic uses. This position detection and/or displacement measurement unit detects, for example, the corresponding position and/or a movement of the anchor means, of an associated valve spool component group or an associated piston rod of a hydraulic cylinder. The position detection and/or displacement measurement nowadays generally takes place by means of inductive sensor technology, such as inductive position switches or inductive displacement sensors, for example by means of LVDT (linear variable differential transformer) sensors, cf. FIG. 3. This allows a contact-free and thus wear-free detection and/or measurement, wherein in particular LVDT sensors enable unambiguous position detection even after a voltage drop because of their absolute measuring principle. Disadvantages of the known sensor technology are the relatively large required installation space of a resulting device and the required relatively intricate shielding against mechanical damage, impurities and aggressive media in the device.

The object of the invention is to overcome or at least significantly lessen the mentioned disadvantages of the state of the art. In particular, an improved electromagnetic positioning device having integrated sensor technology is to be provided, the sensor technology allowing reliable position detection and/or displacement measurement and simultaneously requiring less installation space while also allowing a simplified and preferably inexpensive constructive design of the positioning element. This object is attained by the subject matter of the independent claim(s). The dependent claims represent advantageous variations of the invention at hand. Moreover, the invention addresses further problems, as will be further discussed in the following description.

In a first aspect, the invention relates to an electromagnetic positioning device having energizable stationary coil means, a magnetic guide element associated thereto and having a core section and an anchor unit moveable in relation to the guide element and along an axial movement direction as a reaction to an energization of the coil means, wherein the guide element is a hollow cylinder that at least partially surrounds the anchor unit, the positioning device having an ultra-wide-band sensor unit which is preferably integrated in the guide element and is designed for measuring the position and/or the displacement or detecting the position and/or measuring the displacement of the anchor unit in the guide element.

The invention at hand allows providing an effective and reliable position detection and/or displacement measurement of the anchor unit in the guide element via the integrated ultra-wide-band sensor unit, in particular for state monitoring and/or regulating the electromagnetic positioning device. The ultra-wide-band sensor unit preferably guided in the guide element significantly reduces a required installation space for the device and simultaneously enables a constructively simplified design at a significant cost reduction in particular with respect to state-of-the-art LVDT sensor technology. Moreover, the integrated ultra-wide-band sensor unit can serve for providing further state and working information of the device. Moreover, the invention at hand allows a particularly precise position detection, including in particular a real-time position detection, in contrast to displacement sensors which are known from the state of the art, generally have a hysteresis and thus by necessity lead to a certain imprecision.

An ultra-wide-band sensor unit in this case is a sensor unit which emits electromagnetic waves and functions based on UWB (ultra wide band), i.e., the emitted electromagnetic waves have a particularly large bandwidth, preferably a bandwidth of more than 500 MHz. The ultra-wide-band sensor unit is designed in particular for emitting and detecting a broadband signal at a frequency ranging between 3.1 GHz and 10.6 GHz, more preferably between 100 MHz and 8.5 GHz, even more preferably between 100 MHz and 6 GHz.

In a particularly preferred embodiment, the ultra-wide-band sensor unit functions at a frequency ranging between 100 MHz and 6 MHz having a bandwidth of at least 500 MHz, preferably at least 1 GHz, particularly preferably at least 2 GHz, more preferably at least 4 GHz and particularly preferably at least 5.5 GHz, and/or functions at a frequency ranging between 6 GHz and 8.5 GHz having a bandwidth of at least 500 MHz, preferably 1 GHz, preferably at least 1.5 GHz, more preferably at least 2 GHz and particularly preferably at least 2.5 GHz. This can advantageously avoid an, in particular bidirectional, disturbance via other radio sources, such as LoRa, 5G or WiFi (in particular 802.11p). Advantageously, in particular the high bandwidth of the frequency ranging between 6 GHz and 8.5 GHz allows attaining a particularly high spatial solution and/or a particularly low minimal measuring distance. Moreover, radio licenses, in particular for a transmitting power in range of −41.3 dbm/MHz, are advantageously not required for this frequency range. Preferably, the transmitting power of the ultra-wide-band sensor unit is −41.3 dbm/MHz or less.

In particular, the frequency band between 6 GHz and 8.5 GHz is intended for at least a distance measurement, in particular in the close range of the sensor unit.

In particular it is also conceivable that the sensor unit or the sensor module is designed in such a manner that one sensor or at least two sensors of the sensor unit measure and/or are operated at least partially simultaneously or alternatingly in both frequency bands (100 MHz to 6 GHz and 6 GHz to 8.5 GHz). This advantageously allows combining a close-range distance measurement and an analysis of a contacted fluid, in particular a hydraulic oil, with a sensor unit or to form one sensor module preferably by means of impedance spectroscopy.

In a preferred embodiment, the ultra-wide-band sensor unit is designed as an ultra-wide-band radar sensor unit, in particular as a close-range radar sensor cell. This is preferably designed as an unmodulated continuous wave radar. The close-range or rather short-range radar sensor cell functions according to the known radar principle, according to which a signal transit time of an emitted wave reflected from an object is detected. From a registered time difference between the emitted and received signal, a distance between the object and the sensor unit can be determined. Likewise, the speed and acceleration of the object can be detected here.

In particular, the ultra-wide-band sensor unit detects a reflection signal of the emitted electromagnetic waves for detecting the objects moved in the field of vision of the sensor. Preferably, the ultra-wide-band sensor unit senses a frequency difference of the reflection signal, in particular a distance of the reflecting object being able to be concluded from the frequency difference. Advantageously, by increasing a bandwidth of a measuring signal, a measuring signal duration can be shortened and thus a minimal measuring distance of a sensor can be decreased. Preferably, the ultra-band-width sensor unit can be operated continuously.

Preferably, the ultra-wide-band sensor unit does not send pulse signals, meaning that advantageously, no measuring interruption is required for receiving pulse responses. This can advantageously allow a particularly high measuring speed, which in particular allows a high measuring accuracy and/or a measuring of objects at a particularly high speed.

Alternatively, however, it is also conceivable that the ultra-wide-band sensor unit is operated in a pulsed manner. Advantageously, moreover, an influencing and/or disturbance of other radio transmission methods, in particular of other narrowband radio transmission methods, such as LoRa, 5G or WiFi (in particular 802.11p) can be prevented by using ultra-wide-band sensor technology.

In a preferred exemplary embodiment, the ultra-wide-band sensor technology is based on an M-sequence technology. This advantageously allows a particularly precise speed detection, in particular of high speeds, of objects moved in the field of vision of the sensor, preferably even at particularly small distances between the objects and the sensor. Advantageously, M-sequence signals, in particular in contrast to (UWB) pulse signals and/or (UWB) sinusoidal signals, are of lower noise. Advantageously, M-sequence signals, in particular in contrast to (UWB) pulse signals and/or (UWB) sinusoidal signals, are less prone to disturbance. Advantageously, M-sequence signals, in particular in contrast to (UWB) pulse signals and/or (UWB) sinusoidal signals, cause a slight disturbance to other applications, such as narrowband radio applications such as LoRa, 5G or WiFi (in particular 802.11p). Advantageously, M-sequence signals, in particular in contrast to (UWB) pulse signals and/or (UWB) sinusoidal signals, are only slightly influenced and/or disturbed by signals from other radio sources, such as narrowband radio applications such as LoRa, 5G or WiFi (in particular 802.11p). Advantageously, M-sequence signals enable a simultaneous measurement across an entire (UWB) frequency range of the sensor, meaning several thousand measurements can be enabled per second. M-sequence is a pseudo-random, binary sequence known in the field as maximum length sequence or sequence of maximum length. In particular, the M-sequence presents a pseudo noise sequence. In particular, the M-sequence has a flat frequency spectrum, which preferably resembles a white noise. In particular, the ultra-wide-band sensor unit is intended to generate or emit a signal, in particular a pseudo noise signal, based on and/or realized by an M-sequence. In particular, the M-sequence signal can be generated by means of feedback shifting registers. In particular, the sensor module comprises at least one circuit for generating the M-sequence, which preferably has an n-step shifting register for generating the M-sequence. In particular, the ultra-wide-band sensor unit has an emitting unit, which generates and emits an M-sequence emission signal. In particular, the ultra-wide-band sensor unit comprises a receiving unit, which receives portions of the M-sequence emission signal reflected by the object. In particular, the ultra-wide-band sensor unit comprises an evaluation unit, which evaluates the received reflected M-sequence emission signal and determines at least one distance of the reflecting object therefrom. Advantageously, the measurement and the measured result of the ultra-wide-band sensor unit having the M-sequence technology are at least essentially influenced by dirt and/or sediment layers in the area of the measuring path of the ultra-wide-band sensor unit.

The ultra-wide-band sensor unit can be disposed axially in the guide element. In this context, the sensor unit is preferably disposed in such a manner in the guide element that its signal emission direction is essentially parallel to the axial movement direction of the anchor unit. Signal emission direction is presently understood to be a signal's main direction of beam, which is preferably orthogonal to an emission surface of the sensor unit.

The ultra-wide-band sensor unit is preferably oriented toward a front of the anchor unit regarding its signal emission direction. This signal emission direction is preferably disposed orthogonally to the axial direction of movement of the anchor unit. The sensor unit can detect a position and/or a change in position of the anchor unit in relation to the sensor unit and enable a displacement measurement based thereupon. The sensor unit in this instance is preferably disposed so as to be centered to the anchor unit, i.e., in particular centered regarding a (rotational) longitudinal axis of the anchor unit.

The ultra-wide-band sensor unit can be disposed in an abutment element of the guide element for the anchor unit. The abutment element preferably serves for stroke and/or movement limitation of the anchor unit in the guide element and can be integrally formed with the guide element or be designed to be selectively connected thereto.

In another preferred embodiment, the ultra-wide-band sensor unit is radially disposed in the guide element. In this case, the sensor unit is preferably disposed in such a manner in the guide element that its signal emission direction is oriented essentially orthogonal to the axial movement of the anchor unit. In this instance, the ultra-wide-band sensor unit is preferably oriented toward a structured circumferential or lateral surface of the anchor unit with regards to its signal emission direction. The structured circumferential or lateral surface moveable in relation to the sensor unit when moving the anchor unit can be detected and/or be sampled by the sensor unit in this instance, and a position detection and/or a displacement measurement can take place via the sensor unit based thereon. The structured circumferential or lateral surface can, for example, comprise at least one protrusion and/or recess.

In a preferred embodiment, the ultra-wide-band sensor unit has a fluid-tight cover disposed thereon. The cover is at least partially permeable to the signals of the sensor unit emitted or to be received and can be made of plastic or glass, for example. The cover preferably entirely covers an emission surface of the sensor unit. The cover can preferably be flat with an inner surface of the guide element surrounding the sensor unit and/or oriented towards the anchor unit.

For this purpose, the ultra-wide-band sensor unit is preferably disposed in a radial or axial recess of the guide element.

In a preferred exemplary embodiment, the ultra-wide-band sensor unit is configured for detecting the acceleration and/or speed of the anchor element in the axial movement direction. This allows monitoring and evaluating the dynamic behavior of the positioning device.

The ultra-wide-band sensor unit can further be configured for analyzing a fluid, in particular a hydraulic oil, guided in the guide element by means of impedance spectroscopy. This allows monitoring and evaluating the fluid composition and/or the fluid state. For instance, the fluid can be continuously monitored for undesired components, such as water and/or material abrasion, in this case.

The ultra-wide-band sensor unit can be formed within a fluid guided in the guide element in addition to being configured for detecting and/or analyzing currents. This can allow detailed detection and/or monitoring of the dynamic behavior and/or a detailed state monitoring.

The previously mentioned information on the system states of the positioning device in particular also enables predictive maintenance of the device. The sensor unit preferably has a length and breadth of less than 12 mm and a height of less than 22 mm.

The corresponding functional designs of the ultra-wide-band sensor unit can also be exhibited in one ultra-wide-band sensor unit. In this case, the sensor unit can have, as previously described, for example, one sensor or at least two sensors, which measure and/or are operated at least partially simultaneously or alternatingly in preferred frequency bands, for example 100 MHz to 6 GHz and 6 GHz to 8.5 GHz. This advantageously allows simultaneously measuring a position and/or displacement, for example, in particular via close-range distance measuring, and analyzing an abutting fluid, in particular a fluid composition and/or the fluid state.

In another preferred embodiment, the device has at least two ultra-wide-band sensor units, at least one of which is configured for determining the position and/or measuring displacement of the anchor unit and the second of which has one of the previously mentioned functional designs and is configured particularly preferably at least for analyzing the fluid guided in the guide element. For this purpose, the ultra-wide-band sensor units for determining the position and/or measuring displacement are disposed axially in the positioning device, in particular in a guide cylinder of the guide element. The second ultra-wide-band sensor unit for analyzing the fluid guided in the guide element is preferably disposed radially in the positioning device, in particular in a fluid channel extending away from the guide cylinder.

The guide element is preferably configured for the use in hydraulic or pneumatic positioning tasks. For this purpose, the guide element is preferably realized as a pressure-tight element, in which the anchor unit is mounted so as to be moveable. The guide element can have a fluid channel on one end for being connected to a hydraulic component group, in particular a hydraulic valve, through which the anchor unit extends at least partially.

The guide element preferably has a core section and a brace section as well as an intermediary intermediate section made of non-magnetic material, for example a CuAl alloy or an Al alloy.

The guide element can also merely have a hollow-cylindrical core section, which is associated with the coil means. For this purpose, the core section can be designed to have permanent-magnet means, for instance 1 or 2-pin actuators, provided on the anchor means for selective interaction.

In a preferred embodiment, the anchor unit is designed for being connected to a valve coil component group and/or to a positioning element, such as a pestle element, in particular for engaging in a positioning partner, such as the guide groove of a cam shaft. The anchor unit can have distally disposed connectors comprising, for example, an inner or outer thread. Spring means, which prestress the anchor unit in a predefined position and are moreover disposed in the guide element or are provided externally therefor, can be associated with the anchor unit.

In a preferred embodiment, the positioning device has a control unit which is integrated in or associated with the device and is configured for controlling and evaluating signals of the ultra-wide-band sensor unit. The control unit is preferably further connected to the coil means and/or control unit variably supplying power to the coil means. In a particularly preferred embodiment, the control unit is configured for proportional control of the anchor unit via the coil means and based on the signals provided by the ultra-wide-band sensor unit.

This allows a programmable, precise control of valves or positioning elements to take place, for example.

In another aspect, the invention at hand relates to the usage of the electromagnetic positioning device as described above for positioning a hydraulic or pneumatic valve, in particular a CETOP valve.

In another aspect, the invention at hand relates to the usage of the electromagnetic positioning device as described above for preferably displacement-controlled engagement in a positioning partner, in particular in a guide groove of a cam shaft. The electromagnetic positioning device is designed as a 1 or 2-pin actuator, for example, in this instance. The ultra-wide-band sensor unit according to the invention can in this instance serve in particular for monitoring the position and/or switch monitoring of the positioning device, i.e., for controlling whether a desired switch position of the actuator has actually been taken up.

In another aspect, the invention at hand relates to the usage of the electromagnetic positioning device as described above for measuring the position and/or the displacement of a hydraulic cylinder connected thereto.

Further advantages, features and details of the invention are derived from the following description of a preferred exemplary embodiment and by means of the figures.

FIG. 1 shows a longitudinal cut through a preferred embodiment of an electromagnetic positioning device according to the invention at hand;

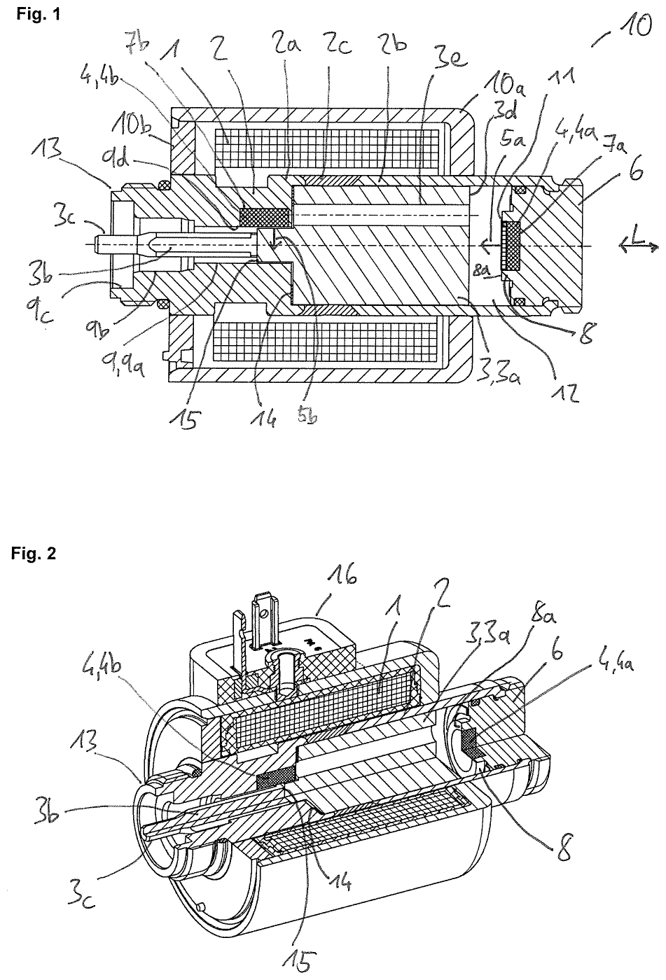

FIG. 2 shows a perspective partial view of the embodiment according to FIGS. 1; and

FIG. 3 shows a perspective partial view of a positioning element from the state of the art having LVDT sensor technology.

FIG. 1 shows positioning device 10 having energizable stationary coil means 1 and a hollow-cylindrical magnetic guide element 2 associated thereto. Coil means 1 are disposed so as to at least partially surround guide element 2. Coil means 1 and guide element 2 extend preferably coaxially within a preferably two-piece casing 10a, 10b of the device. Guide element 2 has a core section 2a, a brace section 2b spaced apart therefrom and an intermediate intermediary section 2c. In guide element 2, anchor unit 3 of device 10 is disposed in such a manner that it can be moved selectively along an axial movement direction L as a reaction to an energization of coil means 1.

Guide element 2 has a hollow-cylindrical tube section 12 and a fluid channel 9 which is connected thereto, is preferably formed coaxially thereto and is connected to a connection area 13 of the device on one end for being connected to, for example, a hydraulic component group. In this instance, fluid channel 9 can have channel sections 9a, 9b, 9c extending gradually towards connection area 13.

Anchor unit 3 comprises a diameter, which is preferably constant having an essentially cylindrical body section 3a, is mounted so as to be moveable in the hollow-cylindrical tube section 12 of guide element 2 and has a fluid channel 3e extending axially through there. Anchor unit 3 further comprises a connective section 3b, which extends away from body section 3a, is preferably disposed coaxially with body section 3a and extends at least partially through fluid channel 9 of guide element 2. Guide channel 2 can have distally disposed connectors 3c, e.g., an inner or outer thread for being connected to an external positioning element.

Guide element 2 can have an abutment element 6 on an end of device 10 opposite connection area 13 on one end, abutment element 6 being designed so as to be connectable integrally or selectively to guide element 3. Abutment element 6 is preferably disposed in guide element 2 as a stroke limitation for anchor unit 3 and can be designed as an essentially cylindrical component, which is disposed in a cylindrical opening of guide element 2. An opposite limitation of the longitudinal movement of anchor unit 3 in guide element 2 can take place by means of an abutment surface 14 integrated in core section 2a of guide element 2.

Positioning device 10 has at least one ultra-wide-band sensor unit 4. This can be disposed axially (cf. reference numeral 4a) or radially (cf. reference numeral 4b) in guide element 2. Ultra-wide-band sensor unit 4 is disposed in an axial or radial recess 7a, 7b of guide element 2 in each instance. When disposed axially, sensor unit 4 is preferably disposed in abutment element 6. In particular, sensor unit 4 can be disposed in a centrally protruding section 8 of abutment element 6. Sensor unit 4 is oriented towards a front face 3d of the anchor unit with regards to its signal emission direction 5a, essentially parallel to axial movement direction L of anchor unit 3. A change in position and/or a displacement measurement along movement direction L can be detected and/or take place via ultra-wide-band sensor unit 4.

When ultra-wide-band sensor unit 4b is disposed radially, sensor unit 4b is preferably integrated in such a manner in fluid channel 9 that its signal emission direction 5b is oriented essentially orthogonal to axial movement direction L of anchor unit 2. The signal emission direction is oriented towards a structured circumferential or lateral surface of anchor unit 3, which can be detected and/or sampled by the sensor when moved along the movement direction. The structured circumferential or lateral surface can, for example, be formed by a circumferential edge 15, which is formed by a change in diameter of anchor unit 3.

Sensor unit 4 can have a cover 11 integrated therewith or associated therewith (shown in an exemplary manner only for axial position 4a). This is preferably disposed flat with an inner surface 8a, 9d of guide element 2 oriented towards anchor unit 3.

As previously described, ultra-wide-band sensor 4a, 4b can also be configured for detecting further parameters. Moreover, the device can also have at least two ultra-wide-band sensor units 4a, 4b, which have the same or different functional configurations for detecting the previously described parameters.

FIG. 2 shows the embodiment according to FIG. 1 in a perspective cut view. In this instance, an integrated connection or a plug unit 16 is shown, which is provided as a connector for coil means 1. This is connected to a control unit (not shown) of device 10, which is also connected to the at least one ultra-wide-band sensor unit 4a, 4b of device 10 and is configured for controlling and evaluating signals of ultra-wide-band sensor 4. The control unit is preferably designed for the proportional control of anchor unit 3 via coil means 1 and based on the signals provided by the at least one ultra-wide-band sensor unit 4a, 4b.

FIG. 3 shows a generic electromagnetic positioning device 10′ having an axially associated LVDT sensor 20 for position detection and/or displacement measurement from the state of the art. LVDT sensor 20 has an oblong encoder core 21, which extends through sensor coils 22 and is disposed in a pressure tube 23. Encoder core 21 is fixedly connected to anchor unit 3′ of the positioning device. A change in position of anchor unit 3′ is detected via a change in relative position of encoder core 21 in relation to associated sensor coils 22.

### REFERENCE NUMERALS

1 coil means
2 guide element
2a core section
2b brace section
2c intermediate section
3 anchor unit
3a body section
3b connection section
3c connector
3d front
3e fluid channel
4 ultra-wide-band sensor unit
4a axial position sensor unit
4b radial position sensor unit
5a,5b signal emission direction
6 abutment element
7a,7b recess
8 protrusion abutment element
8a surface/inner face
9 fluid channel
9a-c fluid channel sections
9d inner face
10 positioning device
10′ state-of-the-art positioning device
10a,b casing parts
11 cover
12 hollow-cylindrical tube section
13 connection area
14 abutment surface
15 structured circumferential or lateral face, edge
16 connection coil means
20 LVDT sensor
21 encoder core
22 sensor coils
23 pressure tube
L movement direction

1. An electromagnetic positioning device having energizable coil means, a magnetic guide element associated thereto and having a core section and an anchor unit moveable in relation to the guide element and along an axial movement direction as a reaction to an energization of the coil means, wherein the guide element is configured to be hollow cylindrical and to at least partially surround the anchor unit, characterized in that the positioning device has an ultra-wide-band sensor unit integrated in the guide element and designed for measuring a position and/or a displacement of the anchor unit in the guide element.

2. The electromagnetic positioning device according to claim 1, wherein the ultra-wide-band sensor unit comprises a short-range radar sensor cell that functions as an unmodulated continuous wave radar.

3. The electromagnetic positioning device according to claim 1, wherein the ultra-wide-band sensor unit is disposed axially in the guide element such that its signal emission direction is essentially parallel to the axial movement direction of the anchor unit.

4. The electromagnetic positioning device according to claim 3, wherein the ultra-wide-band sensor unit is oriented such that its signal emission direction is toward a front of the anchor unit disposed orthogonally to the axial movement direction of the anchor unit.

5. The electromagnetic positioning device according to claim 3, wherein the ultra-wide-band sensor unit is disposed in an abutment element on one end for the anchor unity of the guide element.

6. The electromagnetic positioning device according to claim 1, wherein the ultra-wide-band sensor unit is disposed radially in the guide element, such that its signal emission direction is oriented essentially orthogonally to the axial movement direction of the anchor unit.

7. The electromagnetic positioning device according to claim 6, wherein the ultra-wide-band sensor unit is oriented such that its signal emission direction is towards a structured circumferential or lateral surface of the anchor unit.

8. The electromagnetic positioning device according to claim 1, wherein the ultra-wide-band sensor unit has a fluid-tight cover disposed thereon.

9. The electromagnetic positioning device according to claim 8, wherein the ultra-wide-band sensor unit is disposed in a radial or axial recess of the guide element, and the cover disposed on the sensor unit preferably extends flat with an inner surface of the guide element oriented towards the anchor unit.

10. The electromagnetic positioning device according to claim 1, wherein the ultra-wide-band sensor unit is configured for detecting an acceleration and/or a speed of the anchor unit in the axial movement direction.

11. The electromagnetic positioning device according to claim 1, wherein the ultra-wide-band sensor unit is configured for analyzing a fluid guided in the guide element via impedance spectroscopy.

12. The electromagnetic positioning device according to claim 1, wherein the ultra-wide-band sensor unit is configured for detecting and/or analyzing currents within a fluid guided within the guide element.

13. The electromagnetic positioning device according to claim 1, wherein the anchor unit is configured for being connected to a valve coil component group and/or a positioning element.

14. The electromagnetic positioning device according to claim 1, wherein the guide element has a fluid channel disposed on one end for connecting to a hydraulics component group, through which the anchor unit passes at least partially.

15. The electromagnetic positioning device according to claim 1, wherein the positioning device has a control unit configured for controlling and evaluating signals of the ultra-wide-band sensor unit.

16. The electromagnetic positioning device according to claim 15, wherein the control unit is configured for proportionately controlling the anchor unit via the coil means based on the signals of the ultra-wide-band sensor unit.

17. (canceled)

18. The electromagnetic positioning device according to claim 1, wherein the guide element has a brace section and an intermediary intermediate section made of non-magnetic material in addition to the core section.

19. A method for positioning a hydraulic or pneumatic valve, the method comprising using the electromagnetic positioning device according to claim 1.

20. A method for displacement-controlled engagement in a guide groove of a cam shaft, the shaft method comprising using the electromagnetic positioning device according to claim 1.

21. A method for measuring the position and/or the displacement of a hydraulic cylinder, the method comprising using the electromagnetic positioning device according to claim 1, wherein the hydraulic cylinder is connected to the electromagnetic positioning device.
